(19) United States
(12) Patent Application Publication
(10) Pub. No.: US 2013/0023427 A1
(43) Pub. Date: Jan. 24, 2013

(54) METHODS FOR ASSESSING GENOMIC INSTABILITIES IN TUMORS

(71) Applicants: Anthony P. Shuber, Mendon, MA (US); Thomas C. Meyers, Natick, MA (US); Andy Hu, Lexington, MA (US)

(72) Inventors: Anthony P. Shuber, Mendon, MA (US); Thomas C. Meyers, Natick, MA (US); Andy Hu, Lexington, MA (US)

(73) Assignee: PREDICTIVE BIOSCIENCES INC., Lexington, MA (US)

(21) Appl. No.: 13/364,840

(22) Filed: Feb. 2, 2012

Related U.S. Application Data
(60) Provisional application No. 61/509,898, filed on Jul. 20, 2011.

(51) Int. Cl. G01N 33/574; C40B 30/04; C12Q 1/68

(52) U.S. Cl. 506/9; 435/6.14; 436/501

(57) ABSTRACT

The invention generally relates to methods for assessing genomic instabilities in a tumor sample. The invention may further be used to predict grade, stage, and prognosis of cancer in a patient. The invention further relates to cataloging the efficacy of therapeutics on specific genomic instabilities and generating a personalized therapeutic regimen for a cancer patient based upon their genomic instabilities.

### RELATED APPLICATION

The present patent application claims the benefit of and priority to U.S. Provisional Patent Application Ser. No. 61/509,898 filed on Jun. 20, 2011, the entirety of which is herein incorporated by reference.

### FIELD OF THE INVENTION

The invention generally relates to methods for assessing genomic instabilities in a tumor sample. The invention may further be used to predict grade, stage, and prognosis of cancer in a patient. The invention further relates to cataloging the efficacy of therapeutics on specific genomic instabilities and generating a personalized therapeutic regimen for a cancer patient based upon their genomic instabilities.

### BACKGROUND

Conventional pathology procedures used to detect cancer in biopsy samples involve observing cellular morphology, such as irregular cellular shape, dark dense nuclei, and the like. Cellular features are also used to stage and grade cancer. For example, histologic grade is a determination of how closely the tumor cells resemble normal cells of the same tissue type; while nuclear grade assesses the size and shape of the nucleus in tumor cells and the percentage of tumor cells that are dividing.

All cancers are associated with some form of genomic instability. A primary effort in cancer genetics is directed towards identifying specific genomic abnormalities associated with cancer. The presence of known specific cancer genomic abnormalities in a patient's sample indicates a positive diagnosis for cancer. Generally, diagnostic methods look for genomic instabilities that are known to be associated with cancer and predict grade, stage, and prognosis of the cancer based on the presence or absence of the known genomic instability. Such methods have limited diagnostic value for a large percentage of the population due to the fact that a diagnosis is based upon using only known genomic instabilities and does not account for cancer causing genomic instabilities that have not previously been associated with a particular type of cancer. Additionally, such methodologies treat all genomic instability identically, making a diagnosis based upon presence or absence of the genomic instability while failing to account for differences in those genomic instabilities.

### SUMMARY

The invention provides methods for assessing genomic instabilities in tumor samples in order to predict the grade, stage, and prognosis of cancer. Methods of the invention recognize that all genomic instabilities within a sample are not the same. Genomic instabilities vary in type, location, and size ranging from a discrete mutation in a single base in the DNA of a single gene to a gross chromosome abnormality involving the addition or subtraction of an entire chromosome or set of chromosomes. Moreover, genomic instabilities vary in cancer causing significance and severity. Methods of the invention account for the fact that genomic instabilities vary in cancer causing significance and severity and assess each genomic instability based on its own unique characteristics, which adds a level of information for in-depth prognosis not provided by previous diagnostic and prognostic methods. In this manner, methods of the invention provide a personalized assessment of cancer and therapeutic treatment options for an individual patient.

Methods of the invention account for significance and severity of each genomic instability in a nucleic acid as it relates to causing cancer by assigning a weighted value to each genomically unstable locus in a nucleic acid sequence obtained from a sample. Assigning a weighted value allows methods of the invention to individually assess each genomic instability in the sample and to ultimately causally relate all genomic instabilities within the nucleic acid sequence to an overall grade, stage, and prognosis of cancer for a patient.

The weighted value may be scaled in any manner including and not limited to assigning a positive or negative integer to reflect the significance or severity of the locus as compared to a certain cancer sequence. The weighed value provides significant insight into the prognosis of cancer because each genomically unstable locus may be factored into determining the grade and stage of cancer, instead of only identified matches to known instabilities found in cancer. In one embodiment, a comparison of the weighted values over time and over courses of treatment allows one to alter treatment based on the specific variations of all instabilities linked to cancer.

Methods of the invention provide for assigning weighted values to genomic instabilities within the nucleic acid even if they do not specifically match a genomic instability linked to cancer. Weighted values may also be scaled from a normal reference sequence or from a cancerous reference sequence. In certain embodiments, the weighted values are scaled, factoring in a suspected cancer reference sequence, in which the patient is suspected of having the cancer. Weighted values are assigned to genomically unstable locus based on the type, location, and amount of genetic material affected by the instability.

The genomically unstable loci may be analyzed and characterized using any criteria that allow for grade, stage, and prognosis of cancer to be assessed. For example, each genomically unstable locus may be weighted based on the type of genomic instability found at the locus. Exemplary types of genomic instabilities include subtle sequence changes, alterations in chromosome number, translocations of chromosomes, and gene amplification. Subtle sequence changes and alterations in chromosome number may be further broken down into subtypes including but not limited to additions, deletions, and substitution. Weighted values may be based on solely on the type of change, or the weighted values may be based on comparing the type of genomic instability to a cancer reference.

Another methodology for assigning the weighted value to each genomically unstable locus is based upon a location of the genomically unstable locus within the chromosome. For example, the weighted value may be based upon the proximity of the instability to telomeres. Alternatively, the weighted value may be based upon the proximity of the instability to known locations of genomic instability found in certain cancers. Based on the location, the weighted value is accordingly assigned.

A weighted value may also be assigned to genomically unstable loci within the sample based on the number of nucleotides within each genomically unstable locus. In other words, the amount of genetic material in the nucleic acid affected by the instability determines the assigned weighted value. The amount of nucleotides affected may be subdivided into regions such as but not limited to coding v. non-coding and introns v. exons, and each region assigned a weighted value.

The invention further provides for categorizing the genomically unstable loci and assigning a weighted value for each category. The categories are based on the type, location, and amount of material affected, as described above. After the loci are categorized and assigned a weighted value, a weighted sum may be calculated to represent all of the genomically unstable loci within the sample. In certain embodiments, the weighted sum is the sum of each category's weighted value times the corresponding number of genomically unstable loci in each category. The invention further provides for calculating a weighted average where the weighted sum is divided by the amount of genomically unstable locus in the sample.

Methods of the invention may be used to assess cancer recurrence or progression. After the method is performed in a first sample, the weighted values, sums, or averages are entered into a reference log. The method is then performed again on a second sample from the patient after a lapse in time or course of treatment. The weighted values, sums, or averages of the second sample are also entered into the reference log, and then compared to the first sample. Variances between the first sample's weighted values, sums, or averages and the second sample's weighted values, sums, or averages represents a change in stage or grade of the cancer. Therapeutic treatment may be tailored to the weighted variances providing a specialized treatment based on specific genomic instabilities.

In other aspects, the invention provides methods for quantifying genomic instability. In these aspects, methods of the invention involve sequencing tumor nucleic acid, and comparing the obtained sequence to a reference sequence to determine the number of genomically unstable loci in the sample. The number of genomically unstable loci is then utilized in determining the grade, stage, and prognosis of the cancer for the patient. A biopsy sample is obtained and the nucleic acid is purified and sequenced. Either whole tumor genome sequencing or targeted tumor gene resequencing is performed in order to obtain the sequence. The sample is compared to a reference sequence, such as a human consensus sequence or a non-cancerous sample from the patient, such as a buccal swab. The number of genomically unstable loci is compared to the reference sequence so that the grade, stage, and prognosis of the cancer are determined based upon the quantity of genomic instabilities.

Methods of the invention further provide for integrating the number of genomically unstable loci and a pathology report of the biopsy sample to comprehensively determine the stage, grade, and prognosis of cancer in the patient. Samples are obtained over time to determine a change in the number of genomically unstable loci present in the sample. The number of genomically unstable loci determined over time is compared to a baseline number of genomically unstable loci in order to assess tumor progression, response to treatment, etc.

Any genomically unstable loci are indicative of cancer. Generally, the greater the number of genomically unstable loci the more severe the cancer is, both in terms of prognosis and stage of the cancer.

Methods of the invention further provide for assessing cancer in a patient by obtaining a biopsy sample from the patient and determining a number of genomically unstable loci in the sample. With the same sample, the number of genomically stable loci is also determined and the two numbers are compared to calculate a rational number. A rational number is any number that can be expressed as the quotient or fraction a/b of two integers. The number of genomically stable loci is divided by the number of genomically unstable loci to determine the rational number. The rational number is used to assess the stage, grade, and prognosis of the cancer. The number of genomically stable and unstable loci is determined by whole genome sequencing, targeted gene resequencing, PCR, DNA microarray, fluorescent in situ hybridization, Southern blot analysis, or Northern blot analysis.

Methods of this aspect of the invention also provide for integrating the rational number obtained with a pathology report of the sample to comprehensively determine the stage, grade, and prognosis of cancer in the patient. Over time a subsequent rational number is obtained from a second biopsy sample. Any change in the rational number at a subsequent time point compared to the baseline is a change in the stage, grade, and prognosis of cancer in the patient.

Methods of the invention further provide for assessing the efficacy of a therapeutic treatment for cancer by obtaining a first number of genomically unstable loci from a first biopsy sample then administering a therapeutic treatment to the patient. After the therapeutic treatment is administered, a second number of genomically unstable loci from a second biopsy sample are assessed. The difference in the first number of genomically unstable loci compared to the second number of genomically unstable loci is indicative of determining the efficacy of the therapeutic treatment. If the difference between the first and second number of genomically unstable loci is decreased, the treatment is effective while if there is an increase or no change then that therapeutic treatment is ineffective and an alternate therapeutic treatment should be considered.

Alternatively, the efficacy of the therapeutic treatment can be determined by obtaining a first rational number of the first number of genomically unstable loci compared to a first number of genomically stable loci and a second rational number of the second number of genomically unstable loci compared to a second number of genomically stable loci. The therapeutic treatment is effective if the second rational number is lesser than the first rational number, while if it is greater or there is no change an alternate therapeutic treatment should be considered.

In other embodiments of the invention, a reference library of therapeutic treatments is created and reports the therapeutic treatment's efficacy on specific genomic instabilities. The invention also provides a method for providing personalized therapeutic treatment for a cancer patient by reviewing a pathology report and the number of genomically unstable loci in a biopsy sample. A sample from a cancer patient is obtained and processed to determine the genomically unstable loci in the sample. The sample can be processed by any method, such as sequencing, Northern blot analysis, Southern blot analysis, PCR, fluorescent in situ hybridization, electrophoresis, or DNA microarray. Personalized therapeutic treatment is provided based on both the pathology report and the genomically unstable loci in the sample, where the therapeutic treatment is known to effectively target the genomically unstable loci. Depending upon the genomically unstable loci to be targeted, the therapeutic treatment is altered. Further determinations of different genomically unstable loci results in providing a different therapeutic treatment to the cancer patient, based upon those genomically unstable loci.

### DETAILED DESCRIPTION

Methods of the invention use genomically unstable loci to determine the presence, grade, stage, and prognosis of cancer in a patient. Samples are obtained from a patient suspected of having cancer and the genomically unstable loci are assessed. In certain embodiments, methods of the invention account for significance and severity of each genomic instability in a nucleic acid as it relates to causing cancer by assigning a weighted value to each genomically unstable locus in a nucleic acid sequence obtained from a sample. Assigning a weighted value allows methods of the invention to individually assess each genomic instability in the sample and to ultimately causally relate all genomic instabilities within the nucleic acid sequence to an overall grade, stage, and prognosis of cancer for a patient.

In other embodiments, the invention provides methods for quantifying genomic instability. In these aspects, methods of the invention involve sequencing tumor nucleic acid, and comparing the obtained sequence to a reference sequence to determine the number of genomically unstable loci in the sample. The number of genomically unstable loci is then utilized in determining the grade, stage, and prognosis of the cancer for the patient.

### Obtaining a Tissue Sample

Methods of the invention involve obtaining a sample, e.g., tissue, blood, bone, that is suspected to be cancerous or pre-cancerous. Such samples may include tissue from brain, kidney, liver, pancreas, bone, skin, eye, muscle, intestine, ovary, prostate, vagina, cervix, uterus, esophagus, stomach, bone marrow, lymph node, and blood.

The sample may be obtained by methods known in the art, such as fine needle aspiration, core needle biopsy, vacuum assisted biopsy, direct and frontal lobe biopsy, shave biopsy, punch biopsy, excisional biopsy, or cutterage biopsy.

Once the sample is obtained, nucleic acids are extracted to assess the genomically unstable loci of the sample.

### Extraction

Nucleic acids may be obtained by methods known in the art. Generally, nucleic acids can be extracted from a biological sample by a variety of techniques such as those described by Maniatis, et al., Molecular Cloning: A Laboratory Manual, Cold Spring Harbor, N.Y., pp. 280-281, (1982), the contents of which is incorporated by reference herein in its entirety.

It may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl₃ to denature any contaminating and potentially interfering proteins. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

### Sequencing

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope.™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using a electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

Additional detection methods can utilize binding to microarrays for subsequent fluorescent or non-fluorescent detection, barcode mass detection using a mass spectrometric methods, detection of emitted radiowaves, detection of scattered light from aligned barcodes, fluorescence detection using quantitative PCR or digital PCR methods.

### Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

### Other Methods of the Invention

Other techniques allowing for the detection of a nucleic acid in a sample can be used in the present invention, such as, for example, Northern blot, selective hybridization, the use of supports coated with oligonucleotide probes, amplification of the nucleic acid by RT-PCR, quantitative PCR or ligation-PCR, etc. These methods can include the use of a nucleic acid probe (for example an oligonucleotide) that can selectively or specifically detect the target nucleic acid in the sample. Amplification is accomplished according to various methods known to the person skilled in the art, such as PCR, LCR, transcription-mediated amplification (TMA), strand-displacement amplification (SDA), NASBA, the use of allele-specific oligonucleotides (ASO), allele-specific amplification, Southern blot, single-strand conformational analysis (SSCA), in-situ hybridization (e.g., FISH), migration on a gel, heteroduplex analysis, etc. If necessary, the quantity of nucleic acid detected can be compared to a reference value, for example a median or mean value observed in patients who do not have cancer, or to a value measured in parallel in a non-cancerous sample. Thus, it is possible to demonstrate a variation in the level of expression.

### Determining Presence and Type of Genomically Unstable Loci

As described above, look up tables can be used to compare sequencing results to determine genomically unstable loci of the sequence. Once a genomic sequence from one sample has been determined by sequencing, as described above, hybridization techniques are used to determine variations in sequence between the sample sequence and a reference sequence. The variations between the two sequences are the genomically unstable loci of interest.

The number of genomically unstable loci are quantified for the sample and compared to that of a reference sequence in order to determine stage, grade, and prognosis of cancer in the patient. An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from a non-cancerous tissue taken from the subject, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular cancers can also determine the grade, stage, and prognosis of the cancer. One method of determining the presence of predetermined genomically unstable loci includes PCR. Methods for implementing PCR are well-known. In the present invention, human DNA fragments are amplified using human-specific primers. Amplicon of greater than about 200 bp produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

### Quantitative Assessment of Genomically Unstable Loci

In certain embodiments of the invention, the number of genomically unstable loci is assessed over time. Methods as described above are performed on a second sample obtained from the same subject. The number of genomically unstable loci in the second sample are compared to that of the first to determine stage, grade, and prognosis of cancer in the patient.

In another aspect of the invention, a rational number is determined to assess the stage, grade, and prognosis of cancer in the patient. A rational number is any number that can be expressed as the quotient or fraction a/b of two integers. In this aspect, the number of genomically unstable loci is determined in a sample using methods described above. Further, the number of genomically stable loci from the same sample is determined and a ratio of the number of genomically unstable loci to genomically stable loci provides a rational number for that patient. As discussed above, any genomic instability is indicative of cancer. There is a linear relationship between the rational number and the severity of cancer in the patient, the higher the number, the worse the grade, stage, and prognosis of cancer.

In one embodiment of the invention, a rational number is obtained from the same sample of the subject over time. Any increase in the rational number of genomic instabilities, the more severe the cancer. A rational number that is progressing upwards over time indicates an increasing severity of cancer and ineffectiveness of the current therapeutic treatment. Any decrease in the rational number of genomically unstable loci to genomically stable loci is indicative of the improvement of the grade, stage, and prognosis of cancer for the patient from the previous analysis. Further, the rate at which the rational number changes indicates the severity of the cancer. The more severe the cancer, the steeper the slope will be between the rational number of at least two time points.

Based upon the determination of the number of genomically unstable loci and/or the presence of predetermined genomically unstable loci, methods of the invention also include providing targeted therapeutic treatment based upon the presence and/or quantity of genomically unstable loci in a sample.

### Qualitative Assessment of Genomically Unstable Loci

Certain embodiments of the invention provide for assessing cancer in a patient through a qualitative assessment of genomically unstable loci. The qualitative assessment includes identifying the genomically unstable loci and assigning a weighted value to each genomically unstable locus. Methods of the invention provide for identifying genomically unstable loci by the type of genomic instability, the location of the genomic instability, the amount of genomic material perturbed by the genomic instability, or a combination thereof. Methods of the inventions may be used to profile genomically unstable loci causally related to cancers generally by comparing the patient's sample to multiple cancer references. Alternatively, methods of the invention also provide for profiling genomically unstable loci causally related to a specific cancer that the patient is suspected of having by comparing the genomically unstable loci from a patient's sample to a specific cancer reference. Embodiments further provides for assigning weighted values dependant on the identification step.

The genomically unstable loci can be identified by methods described above. Briefly, look up tables can be used to compare sequencing results to determine genomically unstable loci of the sequence. Once a genomic sequence from one sample has been determined by sequencing, hybridization techniques are used to determine variations in sequence between the sample sequence and a reference sequence. The variations between the two sequences are the genomically unstable loci of interest and the type, location, and amount of genetic material affected can also be identified from the variations.

An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from a non-cancerous tissue taken from the subject, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular cancers can also determine the grade, stage, and prognosis of the cancer. One method of determining the presence of predetermined genomically unstable loci includes PCR. Methods for implementing PCR are well-known. In the present invention, human DNA fragments are amplified using human-specific primers. Amplicon of greater than about 200 by produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

After determining the presence and identity of genomically unstable loci, methods of the invention provide for assigning a weighted value to each genomic instability. The weighted value is based upon a characteristic of the genomic instability, such as the type, location, amount of genetic material affect, or a combination thereof.

Methods of the invention further provide for qualitatively assessing the entire sample with a weighted sum. In such an embodiment, the genomic instabilities are characterized by type, location, or amount of nucleotides affected and each category is assigned a weighted value. A weighted sum is then derived by multiplying each category's weighted value times the number of genomically unstable loci within the category. A weighted average may further be calculated by dividing the weighted sum by a total amount of genomically unstable loci in each category.

In embodiments of the invention, the weighted value may be any integer or identifier based on the significance and severity of the genomically unstable locus. The weighted value acts as a way to scale and score genomically unstable loci in comparison to a normal reference sequence and cancer references. Certain embodiments provide for comparing the sequence to a suspected cancer reference sequence in order to scale the sample sequence in comparison to known instabilities found in the suspected cancer sequence. The invention embodies any method of scoring or scaling.

In certain embodiments, the weighted value for instabilities may be on a scale from −10 to +10. The +10 may indicate the genomically unstable locus is extremely unstable because its an exact match to instabilities found in highly progressed or developed cancers. A +4 may represent a genomically unstable locus that is a latent instability, meaning it will not cause cancer on its own, but may become cancerous upon influence of external factors such as aging and smoking. Whereas +2 may represent a genomically unstable locus found in some undeveloped cancers but nothing directly relates the locus to cancer progression. A 0 on the scale may include instabilities not yet known to have any effect or any negative effect towards cancer. A −10 may include genomically unstable locus shown not to affect cancers, for example the instability relates to learning disabilities. Further, embodiments provide for the weighted scale to include a +1 for loci that are the same as those found in cancer, 0.5 for loci similar to those found in cancer, and 0 for loci without a causal link to cancers.

In certain embodiments, methods of the invention assign a weighted value based upon the type of genomically unstable locus. The main types of genomic instabilities include subtle sequence changes, alterations in chromosome number, translocations of chromosomes, and single nucleotide polymorphisms. It is recognized that genomic instabilities are linked to cancer, specifically genomic instabilities that lead to accumulation of cell death and cell growth. Several articles expand on the types and characteristics of genomic instabilities leading to cancer including Lengauer, Christoph, et. al. “Genetic Instabilities in Human Cancers.” Nature 396 (1998): 643-49; Shen, Zhiyuan. “Genomic Instability and Cancer: An Introduction.” Journal of Molecular Cell Biology (2011).

Genomic subtle sequence changes include additions, deletions, inversions, and substitutions of one or more nucleotides within a sequence, but not to the extent of large chromosomal sequence changes. A single nucleotide polymorphism (SNP) is a type of genomic subtle sequence change that occurs when a single nucleotide replaces another within the sequence. Alterations in chromosome numbers include additions, deletions, inversions and substitutions of chromosomes within a sequence. Chromosome translocation occurs when a segment of a chromosome attaches, or fuses, to another chromosome, or when noncontiguous segments within a single chromosome fuse. The result of chromosome translocation is the fusion of two different genes, in which the fused genes may have cancer causing properties or the translocation results in the disruption or deregulation of normal gene function. Gene amplifications results when multiple copies of a chromosomal segment are reproduced, instead of a single copy.

After identifying the type of genomically unstable locus, methods of the invention provide for assigning a weighted value to each genomically unstable locus. In certain embodiments, if an addition, deletion, substitution, translocation, inversion, amplification, or single nucleotide polymorphism found in the sample is similar or identical to the same type of instability in a cancer, then a weighted value reflecting its significance and severity will be assigned according. For example, consider a nucleic acid sequence with a genomically unstable locus representing an addition X, genomically unstable locus representing a translocation Y, and a genomically unstable locus representing a SNP. Both the addition, the SNP and the translocation are assigned a weighted value. If the addition X in the sample is exactly the same as an addition X found in cancer X, then addition X will receive a high value, such as +10. If the translocation Y is not yet identified as an exact translocation found in cancer sequences, but is very similar to a translocation Z found in a particular cancer, then the value of the instability will be high, such as a +6, but not as high as addition X, which represented an exact match. If the SNP Y is not found in cancers, then its weighted value may be a 0, or if the SNP Y is identified as a harmless SNP then its weighted value will be −8. The assigned values are aggregated to arrive at a score that can be used to predict grade, stage, and prognosis of cancer

Other embodiments assign a weighted value based upon the location of the genomically unstable locus. In one embodiment, the weighted value is assigned based upon determining on which chromosome the unstable locus resides. Different chromosomes have varying functions. Instabilities in certain chromosomes lead to cancer whereas instabilities in other chromosomes have no link to cancer. Therefore, genomic instabilities on a certain chromosome are often indicative of a certain type of cancer, whereas genomic instabilities on other chromosomes have no link to cancer. For example, genomic instabilities associated with chromosome 14 are linked to leukemia. Csinady, et al., Leukemia 23 (2009): 870-76. Genomic instabilities associated with chromosome 10 are linked to brain cancer. Yadav Et Al., JAMA 302.3 (2009): 276-89. Genomic instabilities associated with chromosome 9 are linked to bladder cancer and brain cancer. Schneider, et al., Cancer Genetics and Cytogenetics 191.2 (2009): 90-96 and Simoneau, Marys, Oncogene 19.54 (2000) 6317-323. Genomic instabilities associated with chromosome 4 are not linked to any cancers and are commonly linked to other genetic diseases, such as Huntington and Parkinson. Bernstam, Victor. Handbook of Gene Level Diagnostics in Clinical Practice. CRC, 1992.

An example of assigning weighted values to genomically unstable loci based upon on which chromosome the loci reside is shown here. Consider a sample in which twenty genomic instabilities are found on chromosome 14, five genomic instabilities are found on chromosome 10, and three genomic instabilities are found on each of chromosomes 4 and 9. Using a scale of −10 to +10 for weighted values, genomic instabilities found on chromosome 14 are assigned a value of +10 because chromosome 14 is highly associated with a cancer and in this sample the chromosome had the highest number of genomic instabilities. Genomic instabilities found on chromosome 4 are assigned a value of −10, because instabilities found on chromosome 4 are not generally associated with cancer. Genomic instabilities found on chromosome 9 are assigned a weighted value of 3 because chromosome 9 is associated with a cancer, however, there are only three genomic instabilities on chromosome 9 as compared to chromosome 14 that has twenty genomic instabilities. Similarly, genomic instabilities found on chromosome 10 are assigned a weighted value of 4 because chromosome 10 is associated with a cancer, however, there are only five genomic instabilities on chromosome 10 as compared to chromosome 14 that has twenty genomic instabilities. Based on different values assigned to each genomic instability, it can be determined that the patient most likely has leukemia and is potentially at risk of developing brain or bladder cancer.

Other embodiments assign a weighted value based upon proximity of the genomic instability to known or suspected locations of instabilities in certain cancer. To carry out such methods, identified genomic instabilities are compared to a specific cancer reference, and then weighed according to their locations in regards to the known instabilities that are associated with the specific cancer. For example, consider Cancer X has a genomically unstable locus in the middle of chromosome A, and another genomically instable locus between chromosomes B and C. A sample has a genomically unstable locus in the middle of chromosome A, and a instability near an end of chromosome B. A high weighted value, such as a +10, will be assigned to the locus in the middle of chromosome A because such locus represents an exact match to location of an instability on chromosome X. The instability near the end of chromosome B will have a lower weighted value because it is not an exact match, however the weighted value should reflect the closeness of the genomic instability near the instability between B and C. For example, if the genomically unstable locus is 2 bases away from the cancer causing instability, its weighted value may be an 7, whereas if the genomically unstable locus is 10 bases away, the weighted value may be a 4. The weighed values may then be used to determine the stage and grade of the sample in relation to Cancer X.

Other embodiments assign a weighted value based upon proximity of the genomic instability to the telomeres. Proximity to telomeres is an important characteristic because telomeres and telomerase are linked to cancer. Telomeres are responsible for regulating cell division by capping chromosomes to prevent the ends of intact chromosomes from appearing like DNA breaks to the DNA replication machinery. Telomeres functioning properly prevent chromosomal degradation, fusion, and rearrangements during DNA replication. With normal cell replication, the telomeres begin to shorten until the telomere is gone and the cell dies. However, in many cancerous cells genomic instabilities may prevent the telomeres from getting shorter by initiating an enzyme called telomerase. Telomerase is found in many cancers and allows mutated cancer cells to replicate indefinitely. The following provide more detailed description of telomeres, telomerase, and genomic instabilities De Lange, T. “Telomere-related Genome Instability in Cancer.” Cold Spring Harb. Symp. Quant. Bio. 70 (2005): 197-204, and Greider, Carol, et al. “Telomeres, Telomerase and Cancer.” Scientific American (2009). Genomic instabilities on or near telomeres may further cause various different fusions, additions, deletions, translocations all of which may contribute to cancer. Therefore, location of instabilities near or on telomeres may provide invaluable insight towards identifying the stage and prognosis of cancer in a sequence.

In determining how to weigh the genomically unstable loci near telomeres, many factors may affect the weighted value such as whether the proximity of the genomic instability to the telomere has been linked to cancer in cancer sequences, the potential of the locus in impacting the telomere's function, type of instability and its proximity to the telomere, the amount of genetic material affected by the instability in regards to its proximity to the telomere, and the exact location in regards to the telomere, i.e. on the telomere, a base away from the telomere, or a few bases away from the telomere. For example, Cancer X has a genomically unstable locus residing on a telomere. A sample has a genomically unstable locus two bases away from the telomere. Here, the weighted value may be a 8 because two bases is very close to the telomere and such close proximity may have the potential to impact the telomere's function. In another example, consider that genomically unstable loci located on a first telomere of chromosome A are known to be causally linked to Cancer Y and that genomically unstable loci located on the second telomere of chromosome A have not yet been causally associated with Cancer Y. A sample reference has a genomically unstable locus on the first telomere and a genomically unstable locus on the second telomere. The genomically unstable locus on the first telomere will have a 10 because it represents an exact match to Cancer Y. The genomically unstable locus on the second telomere may have a 7, because its telomere is not yet associated with Cancer Y, but telomeres perform similar functions and its location on the same chromosome may result in the instability having the same cancer causing significance.

In certain embodiments, a weighted value is assigned to a genomic instability based upon the amount of genomic material perturbed by the instability, i.e., the number of nucleotides affected by the instability. A weighted value may be assigned based upon the amount of genetic material affected in the aggregate. In this embodiment, weighted values may be assigned to proportionally reflect the amount of material affected in comparison with other locus. For example, an addition affects 4 bases whereas a translocation affects 10 bases. The weighted value for the addition will be 2 whereas the weighted value for the translocation will be 5. The weighted value of 2 for the addition and the weighted value of 5 for the translocation proportionally and comparatively represent the amount of material affected in each locus.

In another embodiment, the amount of genetic material perturbed by a locus or loci may further be characterized by subdividing the amount of genetic material affected into regions. A single genomic instability may be subdivided into regions, or all of the genomic material affected by all of the genomically unstable locus may be placed into regional categories. The regional divisions may include coding v. non-coding and introns v. exons. A weighted value may be assigned to reflect the amount of genetic material affected in each region. In an example, Cancer X has a known genomically unstable locus affecting 10 nucleotides. A nucleic acid from a sample also has a genomic instability at the same genomically unstable locus that is known to be associated with Cancer X, however, the genomic instability from the sample affects only 3 nucleotides. In this case, the sample genomically unstable locus is assigned a value of 3 to reflect the amount of genetic material affected in comparison to the genomically unstable locus associated with Cancer X. In another example, a genomically unstable locus affects 50 bases in a non-coding region and another genomically unstable locus affects 10 bases in a coding region of chromosome Y. The non-coding region may have a value of 2 because non-coding region mutations do not affect protein function. The coding region in the same sample may have a weighted value of 6, even though less bases were affected, because its function in coding protein carries with it a higher cancer causing potential.

In certain embodiments, more than one characteristic of the genomic instability is assessed to determine the value assigned to that instability. For example, an instability can be assigned a value not only based on its type (e.g., addition, deletion, translocation), but also its proximity to a telomere and its proximity to a known cancer causing genomic instability. In one example, the weighted value for a genomically unstable locus represents the severity of the locus factoring in that the locus is an addition (type) and the addition affects multiple nucleotides (amount of genetic material affected). In such an example, the value reflects two characteristics of the locus. In another example, a weighted value represents that the locus is a gene amplification (type) affecting only a small amount of genetic material (amount of genetic material affected) on a certain chromosome (location). Such example factored in all three characteristics in determining the weighted value.

Another aspect of the invention assesses assessing cancer in a patient by analyzing a nucleic acid from a sample, identifying one or more genomically unstable loci in the nucleic acid, categorizing the genomically unstable loci, assigning a weighted value to each category, and assessing cancer based on the weighted values. The categories include but are not limited to the type of genomic instability, the location of the genomic instability, and the amount of genetic material affected. Applying a weighted value to a category reflects the overall influence of the category containing certain genomic characteristics within the sample.

A method of calculating a weighted sum from the weighted values of the categories is provided here. The weighted sum reflects the overall influence of all of the genomically unstable loci within the sample. A weighted sum may be devised by adding each category's weighted value times the corresponding amount of genomically unstable loci in each category or amount of genetic material affected in each category. For example, category 1 has a weighted value of 10 and contains 2 genomically unstable locus and category 2 has a weighted value of 4 and 1 genomically unstable locus. The corresponding weighted sum equals 24, the result of (10×2)+(4×1). The invention further provides for calculating a weighted average where the weighted sum is divided by the amount of genomically unstable locus in the sample. The weighted average may allow for a more manageable value in the case where weighted sums are extremely large. The weighted average using the above weighted sum equals 8 (the weighted value 24/(2 genomically unstable locus+1 genomically unstable locus).

For example, if the genomically unstable categories are based on type, one sample may include a category of deletion, a category of additions, and a category of gene amplifications. A weighted value for each category may be assigned based on the amount of genomically unstable loci in each category. For example, the weighted value is proportional to the amount of loci in the categories. Take a sample that when categorized by type results in two categories, a deletion category with 7 deletion-type genomically unstable loci and an addition category with 3 addition-type genomically unstable loci. Assigning values to the category's proportionally based on amount results in the deletion category having a weighted value of 7 and the addition category of 3. In another embodiment, a weighted value for a category may be the average of the weighted values for each individual genomically unstable locus. The weighted value for each individual genomically unstable locus is assigned based on the above embodiments of the invention. For example, after categorizing, a sample has a deletion category composed of 2 deletions, deletion A was assigned 8 and deletion B was assigned a 4. The resulting weighted value of the deletion category is 6, calculated by adding the weighted values (8+4=12) divided by the number of weighted values (2).

### Providing and Recording Targeted Therapeutic Treatment Based on Quantitative and Qualitative Assessment

Methods of this invention are useful because the size of the tumor may shrink over the course of treatment while the tumor cells may remain as genomically unstable, if not more so, than when the tumor was a larger size. Alternatively, a tumor may remain the same size in an individual, while the weighted values and, or number of genomically unstable loci may decrease, thus decreasing the stage, grade, and prognosis of cancer in the patient. Therefore, by assessing the presence of specific genomically unstable loci in a sample and/or the quantity of genomically unstable loci, therapeutic treatment can be provided to the patient based on the genomically unstable loci, not only on the presence of a particular type of cancer or location of the cancer in the patient.

An embodiment of the invention includes a reference log based upon the methods of the invention described above and includes the targeted therapeutic treatments provided to patients based upon the number and weighted values of genomically unstable loci in a sample and the efficacy of the therapeutic treatments for the patients in treating the cancer. The reference log contains a total assessment of the genomically unstable loci as compared to a reference sequence and sequences of certain cancers. In certain embodiments, the reference sequence is a normal sequence and the cancer sequence is from a cancer the patient is suspected of having, but the sample may be compared to one or more cancer reference sequences for diagnosis purposes.

After the genomically unstable loci in a first sample are identified, quantified and assigned weighted values and sums based on selected characteristics and categories, the quantity of genomically unstable loci and calculated weighted values and sums are recorded in a reference log for the patient. A second sample from the same patient is taken after a lapse in time, during a course of treatment, or after a course of treatment. Methods of the invention are performed on the second sample to identify, quantify, and assign weighted values and sums to the genomically unstable loci using the same scaling methods and the same characteristics and categories used for sample 1.

The second sample's quantity of genomically unstable loci, weighted values and sums are likewise recorded in the patient's reference log. Variances in the quantity and corresponding weighted values and sums between the two samples represents changes in the stage or grade of the cancer. If the second sample is taken after a course of treatment, the variances among the quantity, weighted values, and sums are indicative of whether the course of treatment is effective. Because the weighted values represent each genomically unstable locus, either individually or categorically, the course of treatment can be specifically tailored to treat genomically unstable loci that are not responding to the treatment. For comprehensive diagnostic treatment methods, the reference log may be used in conjunction with a pathology report. Methods of the invention provide for continuing the method of future samples from the patient after diagnosis and over the course of treatment in order to qualitatively assess the progression or regression of the cancer and to determine an appropriate course of treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of assessing cancer in a patient, the method comprising:
analyzing a nucleic acid from a sample;
identifying one or more genomically unstable loci in the nucleic acid;
assigning a weighted value to each genomically unstable locus; and
assessing cancer based on the weighted values.

2. The method according to claim 1, wherein the weighed value is based on severity of the genomically unstable locus.

3. The method according to claim 1, wherein the weighted value is based on a type of genomic instability.

4. The method according to claim 3, wherein the type of genomic instability is selected from the group consisting of additions, deletions, substitutions, translocations, alterations, amplifications, and single nucleotide polymorphisms.

5. The method according to claim 1, wherein the weighted value is based on a location of the genomically unstable locus.

6. The method according to claim 5, wherein the location is selected from the group consisting of: location on a chromosome, proximity to telomeres, and proximity to known or suspected locations of genomic instabilities found in certain cancers.

7. The method according to claim 1, wherein the weighted value is based on a number of nucleotides within each genomically unstable locus.

8. The method according to claim 1, wherein analyzing comprises sequencing the nucleic acid.

9. The method according to claim 8, wherein sequencing is sequencing-by-synthesis.

10. The method according to claim 8, wherein identifying comprises comparing the sequenced nucleic acid to a reference nucleic acid to thereby identify the genomically unstable loci.

11. The method according to claim 1, wherein prior to the assigning step, the method further comprises categorizing the genomically unstable loci.

12. The method according to claim 11, further comprising deriving a weighted sum for each category.

13. The method according to claim 11, wherein categorizing is based on a type of genomic instability.

14. The method according to claim 11, wherein categorizing is based on a location of the genomically unstable locus.

15. The method according to claim 1, wherein the method is performed again at a later period in time, thereby monitoring progression or recurrence of the cancer.

16. A method of assessing cancer in a patient, the method comprising:
analyzing a nucleic acid from a sample;
identifying one or more genomically unstable loci in the nucleic acid;
categorizing the genomically unstable loci;
assigning a weighted value to each category; and
assessing cancer based on the weighted values.

17. The method according to claim 16, wherein categorizing is based on a type of genomic instability.

18. The method according to claim 16, wherein categorizing is based on a location of the genomically unstable locus.

19. The method according to claim 1, wherein analyzing comprises sequencing the nucleic acid.

20. The method according to claim 10, wherein identifying comprises comparing the sequenced nucleic acid to a reference nucleic acid to thereby identify the genomically unstable loci.

21. The method according to claim 1, wherein the method is performed again at a later period of time, thereby monitoring progression or recurrence of the cancer.

22. A method of assessing cancer in a patient, the method comprising:
analyzing a nucleic acid from a sample;
identifying one or more genomically unstable loci in the nucleic acid;
assigning a weighted value to each genomically unstable locus based on its location; and
assessing cancer based on the weighted values.

23. The method according to claim 22, wherein the location is selected from the group consisting of: location on a chromosome, proximity to telomeres, and proximity to known locations of genomic instabilities found in certain cancers

24. The method according to claim 22, wherein analyzing comprises sequencing the nucleic acid.

25. The method according to claim 24, wherein identifying comprises comparing the sequenced nucleic acid to a reference nucleic acid to thereby identify the genomically unstable loci.

26. The method according to claim 22, wherein the method is performed again at a later period of time, thereby monitoring progression or recurrence of the cancer.

27. A method of assessing cancer in a patient, the method comprising:
analyzing a nucleic acid from a sample;
identifying one or more genomically unstable loci in the nucleic acid;
determining a total number of nucleotides within the genomically unstable loci; and
assessing cancer based on results of the determining step.

28. The method according to claim 27, wherein prior to the assessing step, the method further comprises subdividing the total number of nucleotides within the genomically unstable loci into categories.

29. The method according to claim 28, wherein the categories are selected from the group consisting of: coding v. non-coding and introns v. exons.

30. The method according to claim 27, wherein analyzing comprises sequencing the nucleic acid.

31. The method according to claim 30, wherein identifying comprises comparing the sequenced nucleic acid to a reference nucleic acid to thereby identify the genomically unstable loci.

32. The method according to claim 27, wherein the method is performed again at a later period of time, thereby monitoring progression or recurrence of the cancer.
